(19) United States
(12) Patent Application Publication
(10) Pub. No.: US 2022/0346997 A1
(43) Pub. Date: Nov. 3, 2022

(54) DEVICES, SYSTEMS, AND METHODS FOR OCCLUDING AN ANATOMICAL PASSAGE

(71) Applicant: BOSTON SCIENTIFIC SCIMED, INC., Maple Grove, MN (US)

(72) Inventors: Sean P. Fleury, Princeton, MA (US); Barry Weitzner, Acton, MA (US)

(73) Assignee: BOSTON SCIENTIFIC SCIMED, INC., MAPLE GROVE, MN (US)

(21) Appl. No.: 17/731,704

(22) Filed: Apr. 28, 2022

Related U.S. Application Data
(60) Provisional application No. 63/181,614, filed on Apr. 29, 2021.

(51) Int. Cl. A61F 5/00; A61B 17/12

(52) U.S. Cl. CPC A61F 5/0079; A61F 5/0089; A61B 17/12172; A61B 17/12099; A61B 2017/00606

(57) ABSTRACT

An implantable device capable of regulating flow of material through a body passage. The implantable device may include at least one retention member having a surface curved to resist migration of the implantable device with respect to the body passage. The implantable device may be provided with features providing a cushioning effect with respect to tissue at which the implantable device is implanted. The length of the implantable device and/or the configuration of at least one retention member on the implantable device may be adjustable. A removal element may be provided to facilitate removal of the implantable device.

### PRIORITY

The present application is a non-provisional of, and claims the benefit of priority under 35 U.S.C. § 119 to, U.S. Provisional Application Ser. No. 63/181,614, filed Apr. 29, 2021, the disclosure of which is hereby incorporated by reference herein in its entirety and for all purposes.

### FIELD

The present disclosure relates generally to the field of implantable devices, systems, and methods. More particularly, the present disclosure relates to the field of implantable devices, systems, and methods for regulating or controlling (such as occluding) an anatomical passage. More particularly, the present disclosure also relates to the field of devices, systems, and methods for resisting migration of an implantable device from an anatomical passage.

### BACKGROUND

Various medical treatments involve occluding flow of materials through a body passage. For instance, treatment methods for various medical conditions, such as obesity, diabetes, or duodenal ulcers, involve bypassing the duodenum or restricting flow of materials through the duodenum. If the treatment requires complete bypass of the duodenum, then occlusion (e.g., full occlusion) of the pylorus may be indicated, and an anastomosis may be created, such as between the stomach and the jejunum. A duodenal exclusion device may be placed in the pyloric sphincter to inhibit or block passage of materials (fluid, chyme, etc.) from the stomach through the pylorus and into the duodenum. Various challenges to preventing migration of a deployed occlusion device are presented by the natural movements of the body (e.g., the gastrointestinal system) as well as the constant flow of materials against the occlusion device. Peristaltic movement of the pylorus to pass materials therethrough (e.g., distally into the small intestine), generally less frequent reverse peristalsis through the pylorus (proximally into the stomach), as well as the natural tendency of the pylorus to eject materials therein present particular challenges for placement and retention of pyloric occlusion devices. Moreover, in addition to resisting migration, it may also be desirable for the occlusion device to be removable without damaging the tissue at the deployment site.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an occlusion device is provided with a first retention member, a second retention member, and a saddle between the first retention member and the second retention member.

In accordance with one aspect of the present disclosure, the saddle is configured to be positioned through a body passage, the first retention member and the second retention member are configured to engage anatomical structures on either end of the body passage to inhibit migration of the occlusion device with respect to the body passage, and at least one of the first retention member or the second retention member has a surface curved to resist inversion of the retention member to resist migration of the occlusion device with respect to the body passage.

In some embodiments, the curved surface of the at least one of the first and second retention members is a convex surface. Optionally, the occlusion device is shiftable between a collapsed configuration and an expanded configuration; and the occlusion device further includes a removal element associated with the convex surface of the at least one of the first retention member or the second retention member and configured to shift the occlusion device from an expanded configuration to a collapsed configuration when pulled in a direction away from the convex surface.

In some embodiments, a covering, such as an occlusive covering, is provided on at least one of the first retention member and the second retention member to occlude flow of materials therethrough.

In some embodiments, said occlusion device is shiftable between a collapsed configuration and an expanded configuration, the saddle is configured to be positioned through a pylorus between a stomach and duodenum, the first retention member is an upstream retention member configured to be positioned within the stomach upstream of the pylorus to hold the occlusion device from migrating through the pylorus and into the duodenum, and the second retention member is a downstream retention member configured to be positioned within the duodenum downstream of the pylorus to hold the occlusion device from migrating through the pylorus and into the stomach. In some embodiments, the at least one of the first retention member or the second retention member is the first retention member, the curved surface being a convex surface on an upstream side of the first retention member, facing away from the saddle and toward the stomach, curved to resist to inversion and migration through the pylorus. Optionally, a removal element is associated with the upstream side of the first retention member and configured to return the occlusion device to a collapsed configuration from an expanded configuration when pulled in an upstream direction away from the upstream side of the first retention member. In some embodiments, the upstream retention member has a downstream side, facing the saddle, having a concave surface such that the upstream retention member is resistant to inversion and migration through the pylorus.

In some embodiments, at least one of the first retention member or the second retention member is a double-wall retention member comprising a first wall facing the saddle, and a second wall facing away from the saddle.

In some embodiments, the saddle is adjustable to adjust at least one of: the distance between the first and second walls of a double wall retention member, or the distance between the first and second retention members. Adjustment of the saddle may adjust at least one of the retention members to seat securely with respect to the body passage, such as a pylorus, to occlude flow of materials therethrough.

In some embodiments, the occlusion device includes a barrier between the body passage, such as a pylorus, and a saddle-facing side of at least one of the first retention member or the second retention member to cushion the pylorus.

In accordance with some aspects of the present disclosure, the saddle of the occlusion device is configured to be positioned through a body passage having an inlet and an outlet, at least one of the inlet and the outlet being surrounded by a body wall. In some embodiments, the first retention member is configured to occlude flow of materials therethrough and to inhibit migration of the occlusion device with respect to the body passage. In some embodiments, the second retention member is configured to occlude flow of materials therethrough and to inhibit migration of the occlusion device with respect to the body passage.

In accordance with some aspects of the present disclosure, the saddle of the occlusion device is configured to be positioned through a pylorus; the first retention member is configured to be positioned within a stomach upstream of the pylorus; and the second retention member is configured to be positioned within a duodenum downstream of the pylorus.

In accordance with one aspect of the present disclosure, the occlusion device is configured to occlude flow of material through a body passage having an inlet at an upstream side and an outlet at a downstream side. The first retention member is an upstream retention member configured to occlude the body passage inlet and has an upstream side and a downstream side. The second retention member is a downstream retention member configured to occlude the body passage outlet and has an upstream side and a downstream side.

In some embodiments, the upstream side of the upstream retention member has a convex surface with a convex curvature sufficient to cause the upstream retention member to resist inversion to inhibit downstream migration of the occlusion device through the body passage; and the downstream side of the downstream retention member has a curvature different from the convex curvature of the upstream side of the upstream retention member.

In some embodiments, the downstream side of the downstream retention member is substantially flat or concave or convex with a convex curvature less than the convex curvature of the convex surface of the upstream side of the upstream retention member.

In some embodiments, the downstream side of the upstream retention member has concave surface with a concave curvature sufficient to cause the upstream retention member to resist inversion to inhibit downstream migration of the occlusion device through the body passage.

In some embodiments, the upstream side of the downstream retention member has a convex surface configured to seat around the outlet of the body passage.

In some embodiments, an occlusive covering is provided over the upstream retention member to occlude the flow of materials therethrough.

In some embodiments, an occlusive covering is provided over the downstream retention member to occlude the flow of materials therethrough.

In some embodiments, at least one of the first retention member and the second retention member is a double-wall retention member having a first wall facing the saddle, and a second wall facing away from the saddle. In some embodiments, the saddle is adjustable to adjust the distance between the first wall and the second wall of the double wall retention member.

In some embodiments, the saddle is adjustable in length to adjust the distance between the first retention member and the second retention member.

In accordance with one aspect of the present disclosure, the occlusion device is shiftable between a collapsed configuration and an expanded configuration, the saddle is configured to be positioned through a pylorus between a stomach and duodenum, the first retention member is an upstream retention member configured to be positioned within the stomach upstream of the pylorus to hold the occlusion device from migrating through the pylorus and into the duodenum, and the second retention member is a downstream retention member configured to be positioned within the duodenum downstream of the pylorus to hold the occlusion device from migrating through the pylorus and into the stomach.

In some embodiments, the upstream retention member has an upstream side, facing away from the saddle and toward the stomach, having a convex surface such that the upstream retention member is resistant to inversion and migration through the pylorus. In some embodiments, the pyloric occlusion device also includes an occlusive covering provided over at least the upstream retention member to occlude the flow of materials therethrough.

In some embodiments, an occlusive covering is provided over the downstream retention member to occlude the flow of materials therethrough.

In some embodiments, a removal element is associated with the upstream side of the upstream retention member and is configured to return the pyloric occlusion device to a collapsed configuration from an expanded configuration when pulled in an upstream direction away from the upstream side of the upstream retention member.

In some embodiments, the upstream retention member has a downstream side, facing the saddle, having a concave surface such that the upstream retention member is resistant to inversion and migration through the pylorus.

In some embodiments, at least the downstream retention member is a double-wall retention member including a first wall forming an upstream side of the downstream retention member facing the saddle, and a second wall forming a downstream side of the downstream retention member facing away from the saddle and toward the duodenum. In some embodiments, the saddle is adjustable in length to adjust the distance between the upstream retention member and the downstream retention member to seat the upstream retention member and the downstream retention member securely with respect to the pylorus to occlude flow of materials therethrough.

In some embodiments, the saddle is adjustable to adjust the distance between the first wall of the double wall downstream retention member and the second wall of the double wall downstream retention member.

In some embodiments, the saddle is adjustable in length to adjust the distance between the upstream retention member and the downstream retention member.

In some embodiments, the device further includes a barrier between the pylorus and at least one of the downstream side of the upstream retention member or the upstream side of the downstream retention member to cushion the pylorus.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an occlusion device formed in accordance with various aspects of the present disclosure and positioned in a schematic representation of a gastrointestinal environment.

FIG. 2 illustrates an elevational view of an example of an embodiment of an occlusion device formed in accordance with various aspects of the present disclosure.

FIG. 3 illustrates an elevational view of an example of an embodiment of an occlusion device formed in accordance with various aspects of the present disclosure.

FIG. 4 illustrates a cross-sectional view along line IV-IV of an occlusion device such as illustrated in FIG. 2, showing an example of an adjustment mechanism which optionally is provided in an occlusion device formed in accordance with principles of the present disclosure.

FIG. 5A illustrates a preliminary stage of delivery of an occlusion device formed in accordance with various principles of the present disclosure.

FIG. 5B illustrates delivery of a distal portion of an occlusion device formed in accordance with various principles of the present disclosure

FIG. 5C illustrates delivery of a proximal portion of an occlusion device such as illustrated in FIG. 5D.

FIG. 5D illustrates the occlusion device of FIGS. 5A-5C as deployed.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). The terms “upstream” and “downstream” may be used to refer to the environment in which a device disclosed herein is used (e.g., flow of materials having an upstream direction and a downstream direction), and to describe elements, features, movements, etc., relative to such environment. “Longitudinal” means extending along the longer or larger dimension of an element. “Central” means at least generally bisecting a center point, and a “central axis” means, with respect to an opening, a line that at least generally bisects a center point of the opening, extending longitudinally along the length of the opening when the opening comprises, for example, a tubular element, a strut, a channel, a cavity, or a bore.

In accordance with various principles of the present disclosure, an implantable device is configured to regulate flow of material through an anatomical deployment site (site or location at which the device is positioned/deployed/implanted within a human body) such as a body passage, body lumen, etc. It will be appreciated that terms such as passage, lumen, and the like may be used interchangeably herein without intent to limit, the broad principles of the present disclosure being applicable to various shapes and sizes of body passages, lumens, etc. The body passage may have an inlet and an outlet, and the implantable device may be provided with retention members seated with respect to an anatomical structure along or adjacent the inlet and/or outlet (e.g., a body wall) as anti-migration structures configured to resist migration of the implantable device from the deployment site (e.g., with respect to the body passage). It will be appreciated that terms such as resist, inhibit, prevent (and conjugations thereof) may be used interchangeably herein without intent to limit unless otherwise indicated. It will further be appreciated that the deployment site is generally to be understood as the intended treatment site or position of the device once deployed and in use. The deployment site may be within a body passage extending between anatomical structures (e.g., a body cavity or organ adjacent the body passage) with a diameter generally larger than the body passage. A device deployed in such anatomical configuration may have retention members at either end of a saddle wider (in a radial direction transverse to the longitudinal axis of the body passage) than the saddle (the saddle generally extending along an intermediate region of the device and generally extending through the body passage), and configured to seat against a body wall extending radially outwardly from the body passage.

In some embodiments, if the implantable device is positioned to regulate flow of materials which flow in one general direction more than in another general direction, then the flow stream may be considered to flow in a downstream direction from an upstream direction, the flow stream generally being stronger in the downstream direction than the upstream direction. Directional references are generally made herein to “upstream” and “downstream” for the sake of convenience, and without intent to limit, it being understood that a device formed in accordance with various principles of the present disclosure may be placed in an environment without a flow stream having a particular directionality, or in a relatively static environment without a significant flow of materials through the anatomical structure in which the device is deployed or positioned.

In accordance with an aspect of the present disclosure, the implantable device may be particularly configured to occlude flow of material through an anatomical structure. Such terms as occlude, block, prevent, inhibit, impede, reduce, delay, etc. (and conjugations thereof) may be used interchangeably herein without intent to limit to indicate reduction of flow of materials by greater than 50%, and up to 100% including increments of 1% therebetween. Reference may generally be made to an occlusion device for the sake of convenience and without intent to limit, as it will be appreciated that various principles and aspects of the present disclosure are applicable to implantable devices other than those which specifically occlude flow and/or which reduce flow of materials by less than 50%.

In accordance with a separate and independent aspect of the present disclosure, the device is configured to resist migration from the deployment site of the device. In accordance with a separate and independent aspect of the present disclosure, the device is configured to facilitate removal thereof when desired and/or medically indicated. In accordance with a separate and independent aspect of the present disclosure, the device is configured to be adjustable in the deployment site, such as to adjust its size or position within the deployment site, such as to optimize its fit within the deployment site, such as to optimize regulation of flow therethrough and/or through the deployment site. It will be appreciated that various structures or features provided in connection with such aspects of the present disclosure may be used individually or one or more such structures or features may be used in combination with one another.

A device formed in accordance with various principles of the present disclosure to resist migration may be provided with a retention member shaped and/or configured to engage with one or more anatomical structures at the deployment site to inhibit movement of the device with respect to the deployment site. For instance, a retention member may be in the form of a lateral extension or flange wider than the intermediate region of the device and wider than a body passage in which the intermediate region of the device is deployed, and thus positioned at the inlet (upstream) and/or outlet (downstream) of such body passage and not within such body passage. For the sake of convenience and without intent to limit, references herein to a retention member are generally intended as references to such configuration, although not necessarily so limited as will be appreciated by those of ordinary skill in the art. The device may be provided with a first retention member and a second retention member with a saddle therebetween. The first retention member may be on or adjacent or along or at a first end (in other words, closer to the first end than to the second end) of the device, and/or a second retention member on or adjacent or along or at a second end (in other words, closer to the second end than to the first end) of the device. It will be appreciated that terms such as on or adjacent or along or at may be used interchangeably herein without intent to limit unless otherwise stated, and are intended to indicate a general relative spatial relation rather than a precisely limited location. In some embodiments, at least one of the retention members is configured to resist migration with respect to the deployment site. In some embodiments, the first retention member and the second retention members are shaped similarly, although such shapes may not have the same dimensions (i.e., the shapes may have different relative dimensions, scales, or proportions). For instance, the first retention member and the second retention member may be substantial duplicates of each other, oriented in generally the same direction. Alternatively, in some embodiments, the first retention member and the second retention member may be mirror images (e.g., similar shapes facing in generally opposite directions). In some embodiments, the first retention member and the second retention member are not generally the same or symmetrical. For instance, the first retention member and the second retention member may have different shapes, dimensions, relative proportions, etc.

A retention member may be configured to resist migration by being shaped to resist deformation, such as to resist migration of the device with respect to its deployment site. It will be appreciated that terms such as deformation or flexure or movement or the like (including other grammatical forms and conjugations thereof) may be used interchangeably herein without intent to limit. In some embodiments, a retention member has a wall with at least one side or surface thereof curved with a curvature sufficient to cause the wall to resist flexure such as may result in inversion of the wall. For example, a proximal retention member may have a proximal side with a convex curvature resistant to flexure in a distal direction. More particularly, distal movement of a wall having a surface with a proximally-facing convex curvature generally is resistant to inversion of the proximally-facing convex surface, which requires more force than would be required for distal movement of a wall which is substantially flat or which has a concave proximally-facing surface. Such proximal retention member thus resists proximal movement of the device distally with respect to the deployment site in which the device is positioned. Likewise, a distal retention member may have a distal side with a convex curvature resistant to flexure in a proximal direction. More particularly, proximal movement of a wall having a surface with a distally-facing convex curvature generally requires inversion of the distally-facing convex surface, which requires more force than would be required for proximal movement of a wall which is substantially flat or has a concave distally-facing surface. Such distal retention member thus resists proximal movement of the device proximally with respect to the deployment site. Similar principles apply to the opposite side of each retention member with opposite curvature having a similar effect (a concave curved surface on one side reacting similar to a convex curved surface on another side and vice versa). It will be appreciated that in some embodiments, the intermediate-facing (facing the intermediate portion of the device) surface of the retention member wall may be shaped to optimize seating of the device with respect to the deployment site, such as to conform to the inlet or outlet of a body passage through which the intermediate portion of the device is deployed.

In some embodiments, the retention member is formed with a double wall, with at least one wall being shaped to resist deformation to resist migration of the device. Generally, a double-walled retention member may be considered to have two walls spaced apart from each other, each wall forming a side of the retention member. Each of the double walls of the retention member has an inwardly-directed surface (the surface of one wall facing the other wall) and an outwardly-directed surface (the surface of one wall away from the other wall, and generally along the outer surface of the overall retention member formed of the two walls). Descriptions with respect to a side or surface of a retention member generally may be applied to an outer surface of the wall on such side of the retention member. For instance, descriptions with respect to a proximal side of a retention member may be applied to an outer surface of the proximal wall of a double-walled retention member. Likewise, descriptions with respect to a distal side of a retention member may be applied to an outer surface of the distal wall of a double-walled retention member. Either or both walls of a double-walled retention member may be configured to resist migration of the device in accordance with the above principles.

In some embodiments, only one of the retention members of a device formed in accordance with various principles of the present disclosure has a wall with a convex surface. For instance, distally directed forces on an occlusion device configured for placement within a pylorus may be stronger than proximally directed forces on such device. If it may be desirable to remove such device, the distal retention member may be configured such that the pylorus provides sufficient resistance to proximal movement or migration of a distal retention member of the device. However, upon application of forces greater than naturally occurring proximally-directed forces at the deployment site to remove the device, such a distal retention member may flex to pass through the pylorus. In such embodiment, at least a portion of a proximally-facing surface of the proximal retention member may be convex in a proximal direction and/or a distally-facing surface of the proximal retention member may be concave in a distal direction to inhibit/prevent proximal migration of the device, yet the distal retention member may not include a distally-facing surface that is convex or a proximally-facing surface which is concave. The surfaces of the distal retention member may be substantially flat or planar, or may even be somewhat convex in a proximally-facing direction to facilitate seating against the outlet to the passage through which such a device is positioned (e.g., the pylorus).

A device formed in accordance with various principles of the present disclosure to occlude (understood herein to include fully or substantially fully or even partially occlude, unless otherwise indicated) flow of materials through a body passage includes a proximal end, a distal end, and an intermediate region therebetween, any or all of which regions may be configured to occlude flow of materials therethrough. In some embodiments, the intermediate region (e.g., a saddle) is formed such that materials do not flow therethrough. For instance, the intermediate region may be a substantially solid element, or a tubular element that is constricted or otherwise configured in a known or heretofore known manner to prevent flow of material therethrough.

Additionally or alternatively, any or all of the regions (e.g., at least one retention member and/or a saddle) of the occlusion device may include a covering along or on various portions thereof. As used herein, the term covering is intended to pertain to any of a variety of structures configured to occlude flow of materials therethrough and includes a covering, coating, membrane, sleeve, or the like, including combinations of different types or structures of coverings. Moreover, the term covered (and various grammatical forms and conjugations thereof) may be used interchangeably herein with terms such as coated and the like without intent to limit. Provision of a coating over various portions of a device formed in accordance with various principles of the present disclosure may facilitate removal of the device by inhibiting tissue ingrowth which may impede or otherwise make difficult removal of the device. However, it will be appreciated that selected regions of the device may be left uncovered to promote a degree of tissue ingrowth to facilitate stabilization (e.g., anchoring) of the occlusion device at the deployment site. For instance, the intermediate region of the device (e.g., saddle) and/or at least portions of inwardly facing surfaces (facing towards the intermediate region) of retention members on the device may be at least partially uncoated. Generally, it may be preferable for such uncoated regions to be limited so as not to impede removability of the device should removability be desired or medically indicated.

To facilitate delivery and deployment and optional removal endoscopically or transluminally (or otherwise, without the need for open surgery), a device formed in accordance with various principles of the present disclosure may be configured to shift or move between a collapsed or compact delivery configuration and an expanded deployment configuration, as described in further detail below. In some embodiments, the occlusion device is formed of a plurality of filaments or the like which are braided, woven, knitted, or the like, or of a laser-cut tube (which may in some instances be considered to form a plurality of struts), or otherwise formed to facilitate such ability to shift configurations. It will be appreciated that the term filaments is used for the sake of convenience, and may be used interchangeably herein with such terms as wires or strands or fibers or struts or the like without intent to limit. The device is formed of a biocompatible material (e.g., metal or polymer or alloy), such as a shape memory or heat-formable material which may be advantageously used to facilitate expansion of the device into a desired deployment configuration. Such formation of the walls of a device may leave a plurality of openings therethrough, such as between the filaments or struts or the like. It will be appreciated that the term openings is used for the sake of convenience, and may be used interchangeably herein with such terms as spaces or interstices or the like without intent to limit. A device which is covered to occlude flow of materials therethrough may be covered to prevent flow of materials between such openings in the walls thereof. For instance, a coating applied to the device may be applied to extend through interstices in the walls of the device to prevent flow of material through such interstices. The coating may be formed of a suitable biocompatible material known or heretofore known in the art, such as a polymeric material, such as silicone, polyurethane, polyvinylidene difluoride (PVDF), polytetrafluorethylene (PTFE), Chronoflex®, etc.

A device generally configured to shift from a collapsed or compact delivery configuration to an expanded deployment configuration may be configured to be returned to a collapsed or compact configuration to facilitate removal from the deployment site as desired and/or medically indicated. The shape or configuration or general construction of the device may facilitate shifting to a collapsed or compact configuration in any of a variety of known or heretofore known manners, the precise construction not being critical to the broad principles of the present disclosure. In order to facilitate removal of a removable device, a removal element may be associated with the device (either formed integrally therewith or separately formed and coupled thereto). The removal element preferably is configured to be readily accessible once removal of the device is to be effected. The removal element may be configured to be grasped to cause collapse of the device, such as for proximal removal. In some embodiments, the removal element is a separate loop or an extension of the device (e.g., an extension of wires or filaments forming the device), configured to cause collapse of the device upon proximal retraction.

In accordance with an aspect of the present disclosure which may be separate and independent of any or all of the above-described aspects, various features may be provided on a device to minimize tissue irritation and/or damage and/or potential ulceration. For instance, if a device is configured to occlude flow of materials, constant bombardment of materials against the device and resistance of the device to migration may cause stress on tissue at the deployment site of the device. In some embodiments, a protective barrier may be provided between the device and the tissue at the deployment site. It will be appreciated that the term barrier (in different grammatical forms and conjugations) may be used interchangeably herein with terms such as bumper or cushion or buffer or layer or the like (in different grammatical forms and conjugations), without intent to limit, to indicate a manner of softening or otherwise modifying the device to reduce potential damage or ulceration of tissue contacted by the device, such as for a prolonged period of time. For instance, a protective barrier (e.g., covering, coating, layer, separate element, etc.) may be provided between inwardly facing walls of flanges (facing the intermediate region) of the device and/or along portion of the intermediate region (e.g., a saddle) of the device. In some embodiments, a separate element, such as a washer-type element, may be positioned between the device and the tissue to provide a protective (e.g., cushioning) layer. Such element may have resilient properties (e.g., formed from a shape memory material) allowing for self-expansion from a compact delivery configuration (e.g., sufficiently compact for transluminal delivery). In some embodiments, a coating may be provided to be sufficiently thick to provide a cushioning effect upon the device encountering anatomical structures. A coating may be thickened at least along edges of a device formed in accordance with any of the various principles of the present disclosure (e.g., along points of transition, such as edges or corners of retention members). Increased coating along edges may enlarge the radius of curvature along the edges to form a surface that spreads forces over a larger area of tissue, thereby minimizing effects such as irritation of and/or erosion into the tissue. Such coating can be in addition to or as an alternative to coatings described above. Increased thickness of a coating used to occlude flow of material through the device may also serve to improve encapsulation of the device for such occlusive function and may make the edges of the wires softer (e.g., resiliently compressible or otherwise presenting an atraumatic surface to tissue) than uncoated wires.

Additionally or alternatively, instead of being formed from a metallic material (typically used to form expandable devices such as described herein), the device may be formed from a polymeric material, such a material generally considered less abrasive or presenting lower friction or greater lubricity to tissue encountered by such device. Examples of polymeric materials include PolyFlex™ (TPU95 thermoplastic polyurethane) or another suitable polymeric material known or heretofore known in the art.

In accordance with an aspect of the present disclosure which may be separate and independent of any or all of the above-described aspects, an implantable device such as a flow-regulating/occlusion device may be configured to be adjustable in at least one dimension or region thereof. For example, a device formed in accordance with various principles of the present disclosure has a proximal end and a distal end, and an adjustable intermediate region therebetween. More particularly, the length of the intermediate region may be adjustable. Such adjustability may be appreciated as allowing optimization of the device for variations in anatomy among patients as well as variations or modification of treatments to be effected by use of the device. In some embodiments, the device includes a proximal retention member along a proximal end thereof and/or a distal retention member along a distal end thereof, with an adjustable saddle along the intermediate region of the device therebetween. Adjustability of the saddle may allow the distance between the proximal end and the distal end of the device to be adjusted, such as to adjust the distance between a proximal retention member and a distal retention member. Such adjustability may be advantageous for adjusting a device to the particular anatomy in which the device is to be used, such as to optimize a secure deployment with respect to the anatomical structure along or in which the device is deployed. Additionally or alternatively, if a double-wall retention member is provided, the distance between the walls of such double-wall retention member may be adjustable. For instance, an adjustable saddle may be used to alter the distance between the walls of a double-wall retention member. In some embodiments, altering the distance between walls of a double-wall retention member may affect the tension between such walls, thereby adjusting the retention force provided by such retention member. The distance between the adjustable walls may also be adjusted to reduce tension to facilitate removal of the device.

Various embodiments of implantable devices formed in accordance with various principles of the present disclosure will now be described with reference to examples illustrated in the accompanying drawings. It will be appreciated that various principles of the present disclosure may be applied to occlude flow of materials through other body passages, discussions with respect to the GI system being for the sake of example and illustration and without intent to limit. It will be appreciated that although reference is made herein to an occlusion device, such reference is for the sake of convenience and without intent to limit. Various principles of the present disclosure may be applied to other devices positioned within an anatomical structure, such as a body passage, lumen, body cavity, organ etc., and allowing flow of material (or at least some degree of flow of material) therethrough.

Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present invention is not limited to only the embodiments specifically described herein.

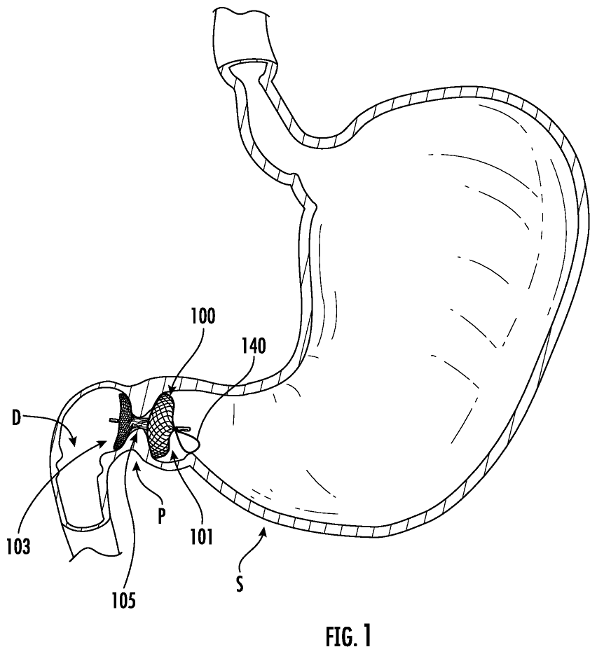

Turning now to the drawings, an example of an implantable device 100, referenced herein as an occlusion device 100 for the sake of convenience and without intent to limit, formed in accordance with various principles of the present disclosure is illustrated in FIG. 1. The illustrated occlusion device 100 has an upstream end 101, a downstream end 103, and an intermediate region 105 therebetween. An upstream retention member 110 may be provided along the upstream end 101 and/or a downstream retention member 120 may be provided along the downstream end 103, with a saddle 130 between the upstream end 101 and the downstream end 103 and along the intermediate region 105. Generally, the saddle 130 is positioned through a body passage, and the retention members 110, 120 are wider than the saddle 130 and positioned against a respective body wall surrounding (e.g., extending outwardly from) a respective inlet and outlet of the body passage to prevent migration of the occlusion device 100 with respect to the body passage (distally/proximally or downstream/upstream of the typical direction of flow of materials through the body passage).

A removal element 140 may be provided on the upstream retention member 110 and/or the downstream retention member 120 to facilitate removal of the occlusion device 100 if desired or medically indicated. The removal element 140 preferably is large enough to be readily grasped by a device inserted in a minimally invasive manner, such as endoscopically or transluminally, and pulled to withdraw the occlusion device 100. In some embodiments, the occlusion device 100 is shiftable between a collapsed delivery configuration and an expanded deployment configuration. Any of a variety of structures may be used, such as known or heretofore known in the art. The removal element 140 may be configured to return the occlusion device 100 to a collapsed delivery configuration suitable for removal in a minimally invasive manner, such as endoscopically or transluminally. For instance, pulling on the removal element 140 may cause the occlusion device 100 to collapse and be withdrawn from the deployment site and move in the direction in which the occlusion device 100 is being pulled. One or more visualization markers, such as radiopaque markers, bands, or radiopaque filler materials, such as known or heretofore known in the art, may be provided to facilitate locating the removal element 140 if and when removal of the occlusion device 100 is desired or indicated.

In accordance with an aspect of the present disclosure, the occlusion device 100 may be configured to regulate, such as to occlude, flow of materials therethrough. As positioned in the schematically represented example of an environment illustrated in FIG. 1 in which the occlusion device 100 may be used, the deployment site of the occlusion device 100 is within the gastrointestinal system. More particularly, the occlusion device 100 may be positioned within or through a pylorus P to regulate flow of gastric materials from the stomach S through the pylorus P and to the duodenum D. However, other uses or environments for an occlusion device 100 formed in accordance with various principles and/or aspects of the present disclosure are within the scope and spirit of the present disclosure. It will be appreciated that reference is made herein to upstream and downstream for directional reference for the sake of convenience and without intent to limit. In general, the upstream end may be considered the end upstream of the general direction of flow of material to be regulated by the occlusion device 100, and the distal end may be considered the end downstream of the general direction of flow of material to be regulated by the occlusion device 100.

Various features of the occlusion device 100 may be shaped and/or configured to impede the flow of materials through the occlusion device 100 and/or through a body passage through which at least a portion of the occlusion device 100 is positioned. For instance, any or all of the surfaces of the occlusion device 100 may be covered or coated with a material preventing flow of materials therethrough. Additionally or alternatively, in some embodiments, the saddle 130 may be solid or, if a tubular element, compressed or otherwise configured to inhibit or prevent flow of materials therethrough. It will be appreciated that in some instance, compressive forces on a solid or compressed saddle 130 will generally have less of an effect on an otherwise collapsible occlusion device 100 such as may occur with (and may even be desirable in) prior devices having retention members 110, 120 which are collapsible and which may be affected, such as induced to collapse, by compressive forces on the saddle.

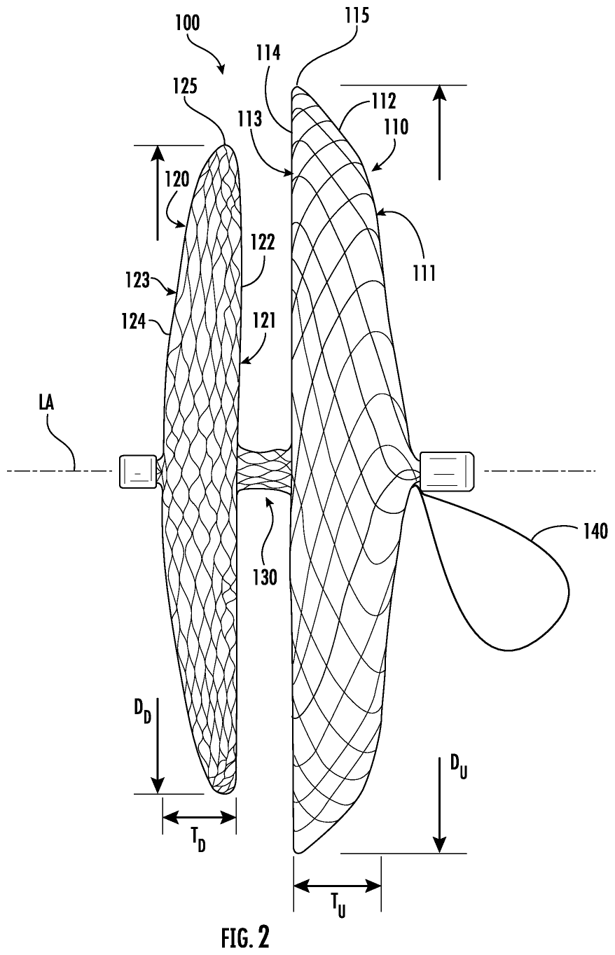

In accordance with an aspect of the present disclosure, as may be appreciated with reference to FIG. 2, at least one of the upstream retention member 110 or the downstream retention member 120 of an occlusion device 100 such as illustrated in FIG. 1 may be shaped and/or configured to resist migration upstream or downstream, such as with respect to the deployment site. In the example of an embodiment illustrated in FIG. 2, the upstream retention member 110 has an upstream side 111 (generally facing away from the intermediate region 105 of the occlusion device 100) with an upstream-facing surface 112 (a portion or the entirety of the upstream side 111) which is convex. As may be appreciated by one of ordinary skill in the art, such convex shape may improve or enhance resistance of the upstream retention member 110 to downstream migration. More particularly, if the upstream retention member 110 is positioned against an anatomical structure such as a pylorus P, the upstream retention member 110 would have to be flexed generally in an downstream direction, such as invert, to move downstream through the pylorus P. A generally convex upstream-facing surface 112 is more resistant to such flexing and inversion than typical retention members which are not generally convex along an upstream side. It will be appreciated that in connection with descriptions herein of inversion of a retention member, various additional factors, such as (and without limitation) the thickness (e.g., thickness T_{U} or thickness T_{D} as illustrated in FIG. 2, generally along a longitudinal axis LA of the occlusion device) of the retention member, the material from which the retention member is formed (e.g., resilient, shape memory, etc.), and general structure of the walls of the retention member (e.g., self-supporting), may contribute to or otherwise affect the resistance to inversion imparted by the shape of a surface of the retention member. As disclosed herein, the shape described as providing an anti-migration effect is generally determinative, without such other factors having an effect. In other words, the wall of the retention member is sufficiently resilient (and not overly thin or flimsy) for the shape to have an effect on the wall, and the wall is configured such that the shape generally controls whether or not the retention member resists migration as desired, a differently shaped wall not resisting migration as would a wall formed in accordance with principles of the present disclosure.

In the example of an embodiment of an occlusion device 100 illustrated in FIG. 2, the downstream side 113 (generally facing the intermediate region 105) of the upstream retention member 110 has a downstream side 113 which may have a downstream-facing surface 114 (a portion or the entirety of the downstream side 113 of the upstream retention member 110) which is concave, substantially straight/planar, or convex depending on various conditions at the deployment site. For instance, a downstream surface 114 having a generally concave shape may enhance resistance of the upstream retention member 110 to downstream migration, such as in a manner described above with respect to an upstream side 111 with a convex upstream-facing surface 112. However, the contour of a surface along one side of the upstream retention member 110 resisting downstream migration may be sufficient. In such instance, if the upstream side 111 has a surface contoured to resist migration sufficiently without the need for contouring the downstream side 113 to resist migration, then the downstream side 113 may be substantially planar (not otherwise distinctly concave or convex curved) or even contoured other than concave. In some embodiments, if desired, the downstream side 113 may be contoured to seat more securely against the inlet to the body passage across which the occlusion device 100 is positioned. For example, the downstream side 113 may have a generally convex downstream-facing surface 114 configured to mate with a generally concave anatomical structure such as a pylorus P. The degree of convexness (the radius of curvature) preferably is selected so as not to affect the anti-migration effect of the convex upstream-facing surface 112 of the upstream retention member 110.

In a similar fashion, in the example of an embodiment of an occlusion device 100 illustrated in FIG. 2, the downstream retention member 120 has a downstream side 123 (generally facing away from the intermediate region 105) which may have a downstream-facing surface 124 (a portion or the entirety of the downstream side 123 of the downstream retention member 120) which is convex in the downstream direction. As may be appreciated by one of ordinary skill in the art, such convex shape may improve or enhance resistance of the downstream retention member 120 to upstream migration. More particularly, if the downstream retention member 120 is positioned against an anatomical structure such as a pylorus P, the downstream retention member 120 would have to be flexed generally in an upstream direction, such as invert, to move upstream through the pylorus P. A generally convex downstream-facing surface 124 is more resistant to such flexing and inversion than typical retention members which are not generally convex along a downstream-facing side.

The upstream side 121 (facing in an upstream direction, generally toward the intermediate region 105) of the downstream retention member 120 may have an upstream-facing surface 122 (a portion or the entirety of the upstream side 121 of the downstream retention member 120) which is concave, substantially straight/planar, or convex depending on various conditions at the deployment site. For instance, an upstream-facing surface 122 having a generally concave shape may add to resistance of the downstream retention member 120 to upstream migration, such as in a manner described above with respect to a downstream side 123 with a convex downstream-facing surface 124. However, if the downstream retention member 120 is positioned against an anatomical structure, such as an outlet to the body passage across which the occlusion device 100 is positioned, the anatomical structure may provide sufficient resistance to upstream migration of the occlusion device 100. In such instance, further contouring of the upstream side 121 of the downstream retention member 120 may serve more to impede removal of the occlusion device 100 than to inhibit or prevent upstream migration (which would be sufficiently addressed by the anatomical structure and positioning of the downstream retention member 120 thereagainst). It may thus be acceptable or even desirable to form the upstream side 121 of the downstream retention member 120 with a substantially straight/planar (e.g., not otherwise distinctly concave or convex curved) upstream-facing surface 112, or even a convex upstream-facing surface 112 which may more readily be seated against the anatomical structure along which the upstream side 121 is positioned.

Variations to and combinations of shapes and/or sizes of retention members 110, 120 of an occlusion device 100 formed in accordance with various principles of the present disclosure may be determined based on the environment in which the occlusion device 100 is to be deployed, anatomy of the patient, the particular needs of the patient, etc. In some embodiments, the upstream retention member 110 and the downstream retention member 120 may be generally mirror images of each other (with the same general configuration with regard to concavity/convexity, though not necessarily the same sizes or dimensions). In some embodiments, the upstream retention member 110 and the downstream retention member 120 may generally be duplicates of each other (with generally the same configuration with regard to convexity or concavity, though not necessarily the same sizes or dimensions). In environments in which forces impacting the occlusion device 100 are not equal in both directions, the upstream retention member 110 and the downstream retention member 120 may be asymmetrical, such as different shapes (e.g., different contours, curvatures, etc.) and/or sizes (e.g., dimensions, proportions, etc.).

In some embodiments, if the occlusion device 100 is positioned to regulate or occlude flow of materials which tends to be stronger in one direction than in another direction, the retention members 110, 120 may have different configurations. For instance, in a flow stream which is stronger in a downstream direction than an upstream direction, the curvature and/or size (overall size or selected dimensions) of the downstream retention member 120 may not be as large as the upstream retention member 110. For example, in the example of an environment illustrated in FIG. 1, the flow of materials (e.g., gastric materials) is stronger in a downstream direction from the stomach S through the pylorus P to the duodenum D. An occlusion device 100 positioned across the pylorus P, such as illustrated in FIG. 1, and formed in accordance with various principles of the present disclosure may have an upstream retention member 110 with an upstream side 111 having a convex upstream-facing surface 112 with a greater degree of curvature (smaller radius of curvature) than the curvature of a downstream-facing surface 124 on a downstream-facing side 123 of the downstream retention member 120. Additionally or alternatively, an occlusion device 100 positioned across the pylorus P, such as illustrated in FIG. 1, and formed in accordance with various principles of the present disclosure may have a proximal retention member 110 that is larger in one or more dimensions than the downstream retention member 120. For instance, as illustrated in FIG. 2, an embodiment of an occlusion device 100 formed in accordance with various principles of the present disclosure (and which may, for example, be used in an environment as illustrated in FIG. 1) may have a proximal retention member 110 with a diameter Du (generally transverse to a longitudinal axis LA of the occlusion device 100) larger than the diameter D_{D} of the downstream retention member 120. Additionally or alternatively, the proximal retention member 110 of an occlusion device 100 such as in the embodiment illustrated in FIG. 2 may have a larger thickness T_{U} (generally along a longitudinal axis LA of the occlusion device 100) than the thickness T_{D} of the downstream retention member 120.

In some embodiments, an occlusion device 100 formed in accordance with various principles of the present disclosure is deployed across a body passage with the upstream retention member 110 and the downstream retention member 120 positioned against tissue walls surrounding a narrower body passage through which the saddle 130 is positioned. In such instance, the body walls surrounding the inlet and outlet to the body passage may provide a sufficient area against which one or both of the upstream retention member 110 and the downstream retention member 120 may be positioned to form an obstacle to migration and have an anti-migration effect. The strength of the body walls and/or the strength of the upstream retention member 110 and downstream retention member 120 to resist flexure or other deformation allowing migration may be sufficient to allow an other-than-concave contour of either face of the upstream retention member 110 and the downstream retention member 120 positioned towards, and generally in apposition with, the body walls surrounding the inlet and outlet through which the saddle 130 is positioned. For instance, in a flow stream which is stronger in a downstream direction than an upstream direction, the body wall surrounding the outlet to the body passage across which the occlusion device 100 is positioned (e.g., the duodenal side of the pylorus P in the example of an environment illustrated in FIG. 1) may provide a degree of resistance to upstream migration of the occlusion device 100. Moreover, if removal of the occlusion device 100 may be desired at some point in time after deployment, and such removal is in an upstream direction, then further resistance to upstream movement of the occlusion device 100 may not be necessary, particularly if the upstream body walls may provide resistance to removal of the downstream retention member 120 in an upstream direction. As such, provision of a concave-curved surface facing the intermediate region 105 of the occlusion device 100 (generally in a direction in which the retention member 110, 120 is provided to inhibit migration) and positioned against a body wall may not be necessary.

It may be desirable for the downstream side 113 of the upstream retention member 110 and/or the upstream side 111 of the downstream retention member 120 (facing the saddle 130) to have a convex surface, particularly in environments in which the upstream retention member 110 and the downstream retention member 120 are positioned against an anatomical structure (e.g., the pylorus P in the example of an environment illustrated in FIG. 1) through which a narrower body passage extends and in which the saddle 130 is positioned. Such configuration may improve seating of the retention member 110, 120 with such convex surface. Improved seating of a retention member 110, 120 may enhance the ability of the occlusion device 100 to occlude flow of materials past the retention member 110, 120 and through the body passage, such as by providing better sealing.

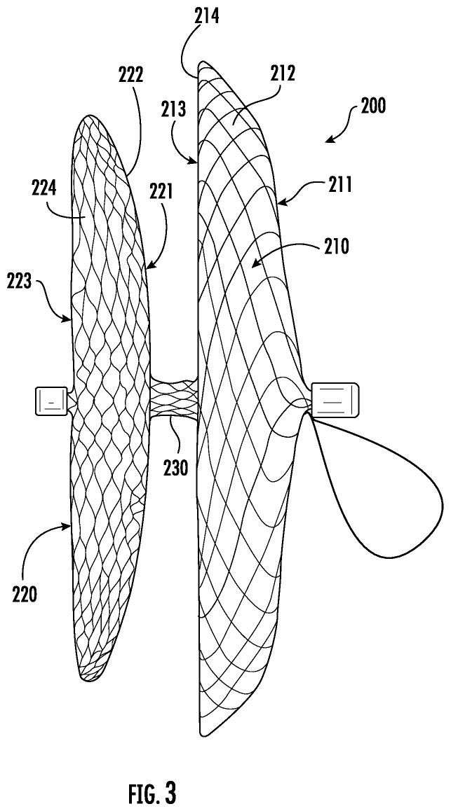

Another example of an occlusion device 200 formed in accordance with various principles of the present disclosure is illustrated in FIG. 3. The structures and features of the occlusion device 200 of FIG. 3 which are similar (other than differences in contours or shapes or sizes) to structures of the occlusion device 100 of FIG. 2 are numbered with the same reference numbers increased by 100. As may be appreciated, the upstream retention member 210 of the example of the occlusion device 200 illustrated in FIG. 3 is substantially similar to the upstream retention member 110 of the occlusion device 100 illustrated in FIG. 2, and thus reference is made to descriptions of the upstream retention member in connection with FIG. 2 for the sake of brevity. The downstream retention member 220 of the example of an occlusion device 200 illustrated in FIG. 3 may differ in configuration from the downstream retention member 120 of the example of an occlusion device 100 illustrated in FIG. 2 in one or more aspects. For instance, the downstream side 223 of the downstream retention member 220 of the occlusion device 200 illustrated in FIG. 3 need not be convex, and optionally may have a substantially flat downstream-facing surface 224 (a portion or the entirety of the downstream side 223 of the downstream retention member 220), such as illustrated. The upstream side 221 of the downstream retention member 220 may have a convex upstream-facing surface 222 (a portion or the entirety of the upstream side 221 of the downstream retention member 220), such as to facilitate seating against a body wall surrounding a body passage through which the saddle 230 is positioned.

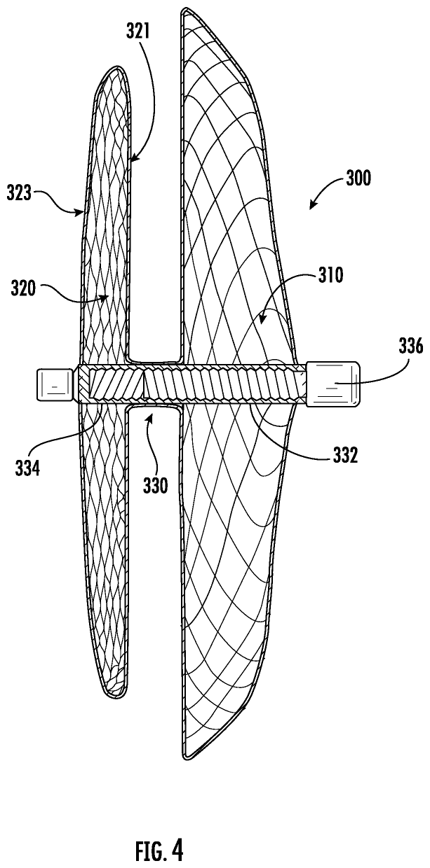

In accordance with a separate and independent aspect of the present disclosure, an example of an occlusion device 300 having an adjustable saddle 330 is illustrated in FIG. 4. The saddle 330 of an occlusion device 300 formed in accordance with various principles of the present disclosure may be adjustable to provide any of a variety of benefits, such as to optimize seating of either or both of the upstream retention member 310 and the downstream retention member 320 along a body wall and/or to optimize the distance between the upstream retention member 310 and the downstream retention member 320 and/or to adjust other properties (e.g., shape, dimension, flexibility, retention strength, etc.) of one or both of the retention members 310, 320. In the example of an embodiment of an adjustable saddle 330 illustrated in FIG. 4, the saddle 330 may include a male threaded member 332 threadedly engaged with a female threaded member 334. An actuator 336, such as a knob, may be provided on an end of one of the threaded members 332, 334 (in the embodiment illustrated in FIG. 4, the male threaded member 332) to facilitate rotation thereof relative to the other of threaded members 332, 334. Relative rotation of the male threaded member 332 and the female threaded member 334 results in the length of the saddle 330 increasing or decreasing depending on the direction of relative rotation. Adjustment of the length of the saddle 330 affects the distance between the upstream retention member 310 and the downstream retention member 320 to adjust to the anatomy of the patient in which the occlusion device 300 is being deployed. Additionally or alternatively, adjustment of the saddle 330 may adjust the distance between walls of a given double-wall retention member. For instance, in the example of an embodiment illustrated in FIG. 4, the female threaded member 334 is coupled to the wall on the downstream side 323 of the downstream double-wall retention member 320 and the male threaded member 332 is coupled to the wall on the upstream side 321 of the downstream double-wall retention member 320. Movement of the male threaded member 332 and the female threaded member 334 closer together brings the walls on the downstream side 323 and the upstream side 321 of the downstream retention member 320 together, to reduce the thickness of the downstream retention member 320, which may decrease the overall flexibility and/or increase the retention strength of the downstream retention member 320. Conversely, movement of the male threaded member 332 and the female threaded member 334 further apart separates the walls on the downstream side 323 and the upstream side 321 of the downstream retention member 320, increasing the thickness of the downstream retention member 320, which may increase the overall flexibility and/or decrease the retention strength of the downstream retention member 320 such as to facilitate removal of the occlusion device 300. It will be appreciated that other configurations allowing for adjustability of a saddle of an occlusion device formed in accordance with various principles of the present disclosure are within the scope and spirit of the present disclosure.

The above-described configurations of retention members are particularly advantageous if provided on an occlusion device 100 which is configured to occlude (e.g., fully occlude) passage of materials therethrough. The impact of forces on an occlusion device 100 may cause the occlusion device 100 to migrate, and various anti-migration features such as configurations of retention member 110, 120 as described above may be particularly advantageous for resisting migration.

In accordance with various aspects of the present disclosure, an implantable device may be formed as an occluding device by being coated with a material which prevents other materials, such as fluids or solid particles, from passing therethrough. As described above, a device in accordance with various principles of the present disclosure may be formed with a plurality of strands or wires or filaments or struts forming a structure which may be shifted between a collapsed delivery configuration and an expanded deployment configuration. Various interstices formed in such construction of an occlusion device may be covered and/or filled by application of a coating over the occlusion device. The coating may be made of silicone and applied in any of a variety of manners to the occlusion device, such as by being sprayed on the occlusion device or by dipping the occlusion device into a silicone bath. Other suitable materials for coating an occlusion device formed in accordance with various principles of the present disclosure include, without limitation, polyurethane, polyvinylidene difluoride (PVDF), polytetrafluorethylene (PTFE), Chronoflex®, or similar biocompatible polymeric formulations.

As noted above, it may be desirable for an occlusion device 100 formed in accordance with various principles of the present disclosure to be removed from the deployment site. Provision of a coating may inhibit tissue ingrowth within the walls of the occlusion device 100 and may facilitate removability of a coated device.

Additionally or alternatively, a protective layer, such as a protective coating, may be applied in varying amounts or thicknesses or configurations, such as to create a barrier between the occlusion device 100 and tissue contacted by the occlusion device 100. For instance, a coating provided over the occlusion device 100 may be sufficiently thick to improve encapsulation of the device for occlusion purposes and additionally provide a degree of resiliency or cushioning against tissue. In some embodiments, the increased thickness in coating may be particularly beneficial along transition areas, such as transitions from the upstream side 111 or the downstream side 113 of the upstream retention member 110 to the longitudinally-extending side 115 of the upstream retention member 110, or from the upstream side 121 or the downstream side 123 of the downstream retention member 120 to the longitudinally-extending side 125 of the downstream retention member 120 (see, e.g., FIG. 2). An increase in coating thickness along transition areas may enlarge the radius of curvature of such areas to spread forces of such areas over a larger area, thereby minimizing any potential undesired effect on the tissue such as erosion. For instance, a generally concave downstream side 113 of an upstream retention member 110 as in the example of an embodiment illustrated in FIG. 2 may be provided with a protective layer to cushion contact with an anatomical structure surrounding a body passage through which the saddle 130 is positioned.

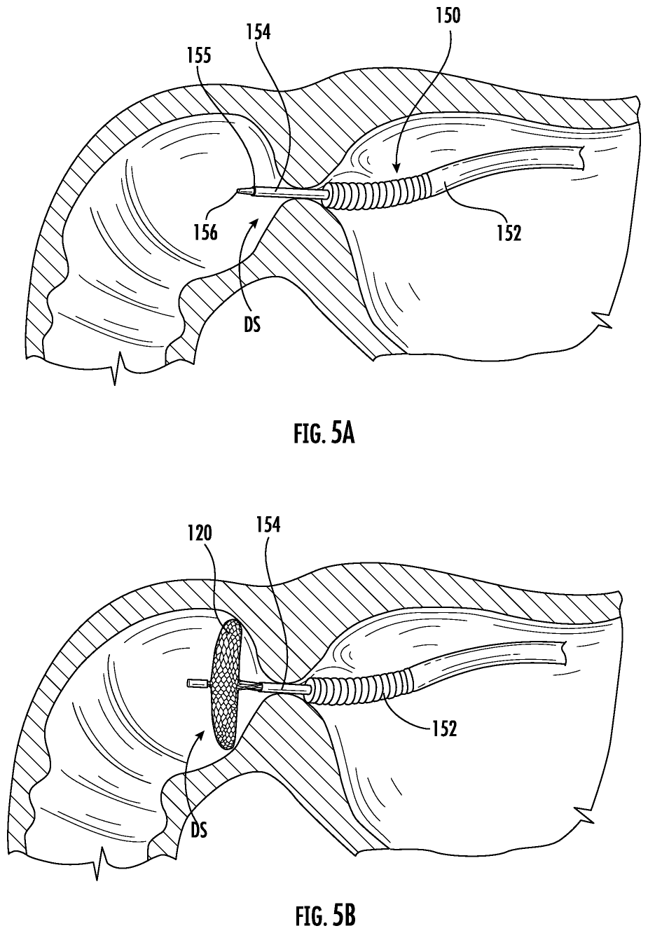

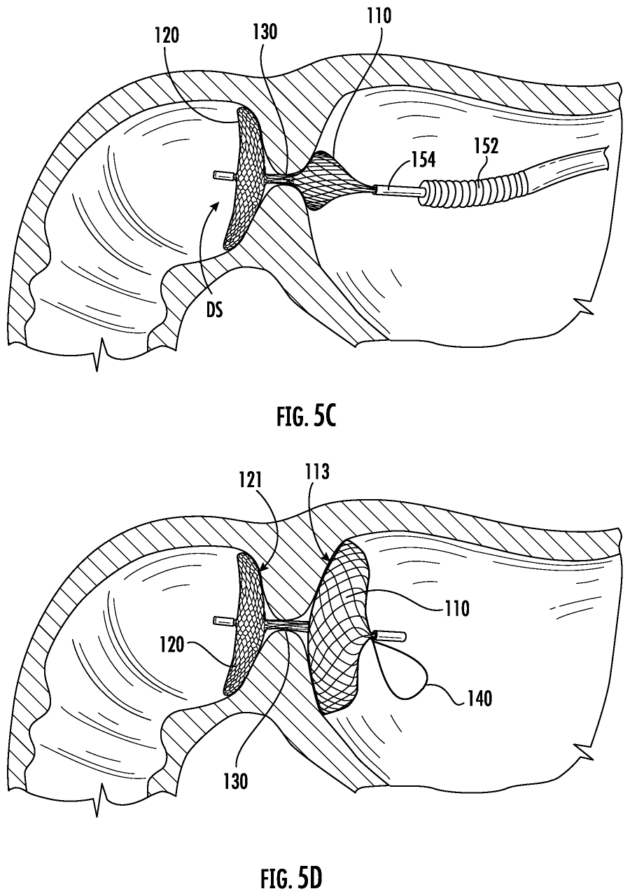

As may be appreciated in view of the above, an occlusion device 100 formed in accordance with various principles of the present disclosure can be deployed and optionally removed in a minimally invasive manner, such as endoscopically or transluminally, as will now be described with reference to examples of deployment stages in an example of a deployment site DS illustrated in FIGS. 5A-5D.

As noted above, an occlusion device 100 formed in accordance with various principles of the present disclosure may be configured to be collapsed into a compact configuration for endoscopic or transluminal (e.g., transcatheter) delivery. In the example illustrated in FIG. 5A, the occlusion device 100 is collapsed within a delivery system 150 capable of readily fitting within and being transported through the body (e.g., transluminally through a passage within the body rather than through an opening surgically created in the body). The delivery system 150 may include a flexible tubular element 152 (such as a catheter or an endoscope or other flexible elongate member with a lumen therethrough) having at least one working channel through which various navigation-assisting instruments or devices (e.g., imaging devices) and/or other devices, tools, instruments, etc. may be delivered to a deployment site. The occlusion device 100 may be delivered (and held in a compact configuration) within a tubular sheath 154 which extends within a working channel of the flexible tubular element 152. The flexible tubular element 152 may be guided to the deployment site DS over a guidewire 156. The open end 155 of the tubular sheath 154 may be positioned at a desired deployment location for the downstream end 103 of the occlusion device 100, as illustrated in FIG. 5A, either by being extended distally from the open end 153 of the flexible tubular element 152, or by advancing the open end 153 of the flexible tubular element 152 to the desired deployment location and then retracting the flexible tubular element 152 to leave the tubular sheath 154 in place at the desired deployment location.

Once the tubular sheath 154 is positioned at the desired deployment site DS with the occlusion device 100 therein, the tubular sheath 154 may be withdrawn (e.g., proximally) to unsheathe the downstream end 103 of the occlusion device 100 at the deployment site DS. If the occlusion device 100 has a self-expanding downstream retention member 120, then withdrawal of the tubular sheath 154 allows the downstream retention member 120 to expand into an expanded deployed configuration such as illustrated in FIG. 5B.

As the tubular sheath 154 is retracted (e.g., further proximally), the intermediate region 105 (e.g., saddle 130) of the occlusion device 100 is positioned through a body passage, as illustrated in FIG. 5C. Optionally, the downstream retention member 120 may be pulled proximally into apposition with the outlet to the body passage through which the saddle 130 is positioned. Further retraction of the tubular sheath 154 unsheathes the upstream end 101 of the occlusion device 100 as well, as illustrated in FIG. 5C and FIG. 5D. If the occlusion device 100 has a self-expanding upstream retention member 110, withdrawal of the tubular sheath 154 allows the upstream retention member 110 to expand into an expanded deployed configuration such as illustrated in FIG. 5D.

The example of an occlusion device 100 shown being deployed in FIGS. 5A-5C is illustrated in a fully deployed configuration in FIG. 5D. A removal element 140 may be provided on the upstream retention member 110. Either or both the upstream retention member 110 and the downstream retention member 120 may flex so that the inwardly-facing sides 113, 121 flex to seat against tissue wall at the deployment site DS. As may be appreciated, proximal pulling on the removal element 140 causes the upstream retention member 110 to return to a collapsed configuration, such as a configuration as in FIG. 5C showing the upstream retention member 110 in an intermediate configuration between a collapsed configuration and an expanded configuration.

It will be appreciated that various structures and features of the embodiments described herein and illustrated in the figures have several separate and independent unique benefits. Therefore, the various structures and features described herein need not all be present in order to achieve at least some of the desired characteristics and/or benefits described herein. Moreover, the various features described herein may be used singly or in any combination. Therefore, the present invention is not limited to only the embodiments specifically described herein. The above descriptions are of illustrative examples of embodiments only, and are not intended as limiting the broader aspects of the present disclosure which may be applied to environments other than the gastrointestinal system and to devices other than occlusion devices.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An occlusion device having a first end, a second end, and an intermediate region therebetween, said occlusion device comprising:
a first retention member;
a second retention member; and
a saddle between said first retention member and said second retention member;
wherein:
said saddle is positionable through a body passage;
said first retention member and said second retention member are configured to engage anatomical structures on either end of the body passage to inhibit migration of said occlusion device with respect to the body passage; and
at least one of said first retention member or said second retention member has a side having a surface curved to resist inversion of said retention member to resist migration of said occlusion device with respect to the body passage.

2. The occlusion device of claim 1, wherein:
said occlusion device is shiftable between a collapsed configuration and an expanded configuration;
said curved surface is a convex surface on at least one of said first retention member or said second retention member facing away from said saddle; and
said occlusion device further comprises a removal element associated with said convex surface of said at least one of said first retention member or said second retention member and configured to shift said occlusion device from an expanded configuration to a collapsed configuration when pulled in a direction away from said concave surface.

3. The occlusion device of claim 1, wherein an occlusive covering is provided on at least one of said first retention member and said second retention member to occlude flow of materials therethrough.

4. The occlusion device of claim 1, wherein:
the body passage is a pylorus;
said saddle is configured to be positioned through the pylorus;
said first retention member is configured to be positioned within a stomach upstream of the pylorus;
said second retention member is configured to be positioned within a duodenum downstream of the pylorus; and
said at least one of said first retention member or said second retention member is said first retention member, said curved surface being a convex surface on an upstream side of said first retention member, facing away from said saddle and toward said stomach, curved to resist to inversion and migration through the pylorus.

5. The occlusion device of claim 1, wherein at least one of said first retention member or said second retention member is a double-wall retention member comprising a first wall facing said saddle, and a second wall facing away from said saddle.

6. An occlusion device configured to occlude flow of material through a body passage having an inlet at an upstream side and an outlet at a downstream side, said occlusion device comprising:
an upstream retention member configured to occlude the body passage inlet and having an upstream side and a downstream side;
a downstream retention member configured to occlude the body passage outlet and having an upstream side and a downstream side; and
a saddle between said upstream retention member and said downstream retention member;
wherein:
said upstream side of said upstream retention member has a convex surface with a convex curvature sufficient to cause said upstream retention member to resist inversion to inhibit downstream migration of said occlusion device through the body passage; and
said downstream side of said downstream retention member has a curvature different from the convex curvature of said upstream side of said upstream retention member.

7. The occlusion device of claim 6, wherein said downstream side of said downstream retention member is substantially flat or concave or convex with a convex curvature less than the convex curvature of said convex surface of said upstream side of said upstream retention member.

8. The occlusion device of claim 6, wherein said downstream side of said upstream retention member has a concave surface with a concave curvature sufficient to cause said upstream retention member to resist inversion to inhibit downstream migration of said occlusion device through the body passage.

9. The occlusion device of claim 8, wherein said upstream side of said downstream retention member has a convex surface configured to seat around the outlet of the body passage.

10. The occlusion device of claim 6, wherein an occlusive covering is provided over said upstream retention member to occlude the flow of materials therethrough.

11. The occlusion device of claim 10, wherein an occlusive covering is provided over said downstream retention member to occlude the flow of materials therethrough.

12. The occlusion device of claim 6, wherein at least one of said first retention member and said second retention member is a double-wall retention member comprising a first wall facing said saddle, and a second wall facing away from said saddle.

13. The pyloric occlusion device of claim 12, wherein said saddle is adjustable to adjust the distance between the first wall of said double wall retention member and the second wall of said double wall retention member.

14. The pyloric occlusion device of claim 13, wherein said saddle is adjustable in length to adjust the distance between said first retention member and said second retention member.

15. A pyloric occlusion device shiftable between a collapsed configuration and an expanded configuration, said pyloric occlusion device comprising:
a saddle configured to be positioned through a pylorus between a stomach and duodenum;
an upstream retention member configured to be positioned within the stomach upstream of the pylorus to hold said occlusion device from migrating through the pylorus and into the duodenum; and
a downstream retention member configured to be positioned within the duodenum downstream of the pylorus to hold said occlusion device from migrating through the pylorus and into the stomach;
wherein:
said upstream retention member has an upstream side, facing away from said saddle and toward said stomach, having a convex surface such that said upstream retention member is resistant to inversion and migration through the pylorus; and
an occlusive covering is provided over at least said upstream retention member to occlude the flow of materials therethrough.

16. The pyloric occlusion device of claim 15, wherein an occlusive covering is provided over said downstream retention member to occlude the flow of materials therethrough.

17. The pyloric occlusion device of claim 15, further comprising a removal element associated with said upstream side of said upstream retention member and configured to return said pyloric occlusion device to a collapsed configuration from an expanded configuration when pulled in an upstream direction away from said upstream side of said upstream retention member.

18. The pyloric occlusion device of claim 15, wherein said upstream retention member has a downstream side, facing said saddle, having a concave surface such that said upstream retention member is resistant to inversion and migration through the pylorus.

19. The pyloric occlusion device of claim 15, wherein said saddle is adjustable in length to adjust the distance between said upstream retention member and said downstream retention member to seat said upstream retention member and said downstream retention member securely with respect to the pylorus to occlude flow of materials therethrough.

20. The pyloric occlusion device of claim 15, wherein said occlusion device further comprises a barrier between the pylorus and at least one of said downstream side of said upstream retention member or said upstream side of said downstream retention member to cushion the pylorus.
